(19) United States
(12) Patent Application Publication
(10) Pub. No.: US 2024/0201287 A1
(43) Pub. Date: Jun. 20, 2024

(54) DETECTION METHOD, DETECTION SYSTEM AND RECORDING MEDIUM

(71) Applicant: Mitsubishi Electric Corporation, Tokyo (JP)

(72) Inventor: Kazuki YAMAUCHI, Chiyoda-ku, Tokyo (JP)

(73) Assignee: Mitsubishi Electric Corporation, Tokyo (JP)

(21) Appl. No.: 18/287,023

(22) PCT Filed: Apr. 22, 2021
(86) PCT No.: PCT/JP2021/016362

§ 371 Date: Oct. 16, 2023

(51) Int. Cl. G01R 33/12; G01N 33/543

(52) U.S. Cl. CPC G01R 33/1276; G01N 33/54333; G01N 33/54373

(57) ABSTRACT

A detection method for detecting a detection target magnetic particle using an AC excitation magnetic field, the detection method includes: acquiring a Neel relaxation curve indicating a relationship between a Neel relaxation time and a particle diameter for candidate magnetic particles; acquiring a Brownian relaxation curve indicating a relationship between a Brownian relaxation time and the particle diameter for the candidate magnetic particles; specifying a particle diameter corresponding to an intersection of the Neel relaxation curve and the Brownian relaxation curve as an intersection particle diameter; and selecting a candidate magnetic particle having a particle diameter larger than the intersection particle diameter as the detection target magnetic particle.

### TECHNICAL FIELD

The present disclosure relates to a detection method, a detection system, a program, and a recording medium for detecting a magnetic particle.

### BACKGROUND ART

In recent years, a magnetic immune inspection using a magnetic particle has been developed as a new immune serum inspection. The magnetic immune inspection has an advantage that a washing process required for a conventional immune test represented by a fluorescence type is not required and sensitivity is high. Furthermore, from transparency of a magnetic signal to a human body, it is expected that the magnetic immune inspection is applied to an internal body diagnosis without taking out an inspection object.

In the magnetic immune inspection, a substance such as a protein that binds to a target substance by an antigen-antibody reaction is previously attached to the magnetic particle, so that an amount and position of the target substance can be specified based on the magnetic signal from the magnetic particle.

Japanese Patent Laying-Open No. 2013-228280 (PTL 1) describes a magnetic immune inspection method and an inspection device using an alternating magnetic field. In the inspection method described in PTL 1, the magnetic particle (hereinafter, the particles are referred to as “bound particle”) bound to the target substance is precipitated by a permanent magnet, and only the magnetic particle (hereinafter, the particle is referred to as “unbound particle”) that exists in a supernatant and are not bound to the target substance is excited to acquire a magnetic signal from the unbound particle. The amount of bound particle is indirectly detected by taking a difference between the acquired magnetic signal and the magnetic signal from the magnetic particle in the sample not containing the target substance at all.

### CITATION LIST

### Patent Literature

PTL 1: Japanese Patent Laying-Open No. 2013-228280

### Non Patent Literature

NPL 1: R. Matthew Ferguson and 2 other persons, “Optimization of nanoparticle core size for magnetic particle imaging”, J. Magn. Magn. Mater., 321 (2009), pp 1548-1551

### SUMMARY OF INVENTION

### Technical Problem

In the technique described in PTL 1, detection accuracy is lower than that of direct quantitative inspection because of indirect quantitative inspection. Furthermore, because the bound particle and the unbound particle are required to be separated using the permanent magnet, the technique described in PTL 1 cannot be applied to an in-vivo inspection in which the inspection object is not taken out of the body.

The present disclosure has been made to solve the above problems, and an object of the present invention is to provide a detection method, a detection system, a program, and a recording medium that can be applied to the in-vivo inspection and accurately detect the bound particle.

### Solution to Problem

A detection method according to one aspect of the present disclosure detects a detection target magnetic particle using an AC excitation magnetic field. The detection method includes: acquiring a first curve indicating a relationship between a Neel relaxation time and a particle diameter for candidate magnetic particles; acquiring a second curve indicating a relationship between a Brownian relaxation time and the particle diameter for the candidate magnetic particles; specifying a particle diameter corresponding to an intersection of the first curve and the second curve as an intersection particle diameter; and selecting a candidate magnetic particle having a particle diameter larger than the intersection particle diameter as the detection target magnetic particle.

A detection system according to one aspect of the present disclosure detects a detection target magnetic particle using an excitation magnetic field. The detection system includes a processor to execute information processing for selecting a detection target magnetic particle from candidate magnetic particles. The processor acquires a first curve indicating the relationship between a Neel relaxation time and the particle diameter for the candidate magnetic particles, and acquires a second curve indicating the relationship between a Brownian relaxation time and the particle diameter for the candidate magnetic particle. Furthermore, the processor specifies a particle diameter corresponding to an intersection of the first curve and the second curve as an intersection particle diameter, and selects a candidate magnetic particle having a particle diameter larger than the intersection particle diameter as the detection target magnetic particle.

A computer program according to an aspect of the present disclosure supports a detection system detecting a detection target magnetic particle using an excitation magnetic field. The computer program causes a computer to execute: acquiring a first curve indicating a relationship between a Neel relaxation time and a particle diameter for candidate magnetic particles; acquiring a second curve indicating a relationship between a Brownian relaxation time and a particle diameter for the candidate magnetic particles; specifying a particle diameter corresponding to an intersection of the first curve and the second curve as an intersection particle diameter; and selecting a candidate magnetic particle having a particle diameter larger than the intersection particle diameter as the detection target magnetic particle.

A computer-readable recording medium according to one aspect of the present disclosure records the computer program described above.

### Advantageous Effects of Invention

In accordance with the present disclosure, the phase of the magnetic signal from the detection target magnetic particle having the particle diameter larger than the intersection particle diameter corresponds primarily to the Brownian relaxation time. The Brownian relaxation time varies depending on the presence or absence of the binding between the detection target magnetic particle and the target substance. Consequently, when the excitation magnetic field is applied to the inspection object in which the bound particle and the unbound particle exist, the bound particle can be accurately detected from a difference in the Brownian relaxation time. Furthermore, the bound particle and the unbound particle are not required to be separated, so that the present invention can also be applied to in-vivo inspection.

### BRIEF DESCRIPTION OF DRAWINGS

FIG. 1 is a view illustrating an example of an overall configuration of a detection system according to a first embodiment.

FIG. 2 is a perspective view illustrating a part of the detection system.

FIG. 3 is a view illustrating an example of a hardware configuration of an information processing device.

FIG. 4 is a flowchart illustrating a method for detecting a magnetic particle of the first embodiment.

FIG. 5 is a view illustrating examples of a Neel relaxation curve and a Brownian relaxation curve.

FIG. 6 is a view illustrating an example of effective relaxation curves of bound particles and unbound particles.

FIG. 7 is a view illustrating another example of the effective relaxation curves of the bound particles and the unbound particles.

FIG. 8 is a flowchart illustrating a flow of a subroutine of step S8 in FIG. 4.

FIG. 9 is a view illustrating processing contents of steps S83, S84.

FIG. 10 is a flowchart illustrating a flow of a subroutine of step S10 in FIG. 4.

FIG. 11 is a view illustrating an example of an overall configuration of a detection system according to a second embodiment.

FIG. 12 is a flowchart illustrating a flow of a subroutine of step S8 in FIG. 4 in the second embodiment.

FIG. 13 is a flowchart illustrating a flow of processing of a detection method in a third embodiment.

FIG. 14 is a view illustrating an example of a device executing step S11.

### DESCRIPTION OF EMBODIMENTS

With reference to the drawings, embodiments of the present disclosure will be described in detail below. In the drawings, the same or corresponding part is denoted by the same reference numeral, and the description thereof will not generally be repeated. In the following drawings, a relationship between sizes of components may be different from the actual relationship.

### First Embodiment

### (Entire Configuration of Detection System)

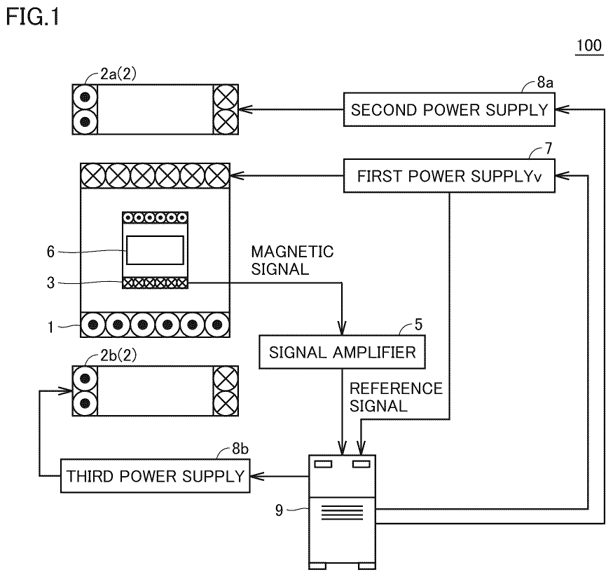

FIG. 1 is a view illustrating an example of an overall configuration of a detection system according to a first embodiment. A detection system 100 in FIG. 1 includes an excitation magnetic field applicator 1, a zero magnetic field generator 2, a magnetic sensor 3, a signal amplifier 5, a first power supply 7, a second power supply 8a, a third power supply 8b, and an information processing device 9.

Excitation magnetic field applicator 1 applies an AC excitation magnetic field to a region where an inspection object 6 is placed. Specifically, excitation magnetic field applicator 1 is configured of a coil connected to first power supply 7. When current flows from first power supply 7 to excitation magnetic field applicator 1, the excitation magnetic field is applied to the region where inspection object 6 is placed.

When the excitation magnetic field is applied to inspection object 6, the magnetic particle included in inspection object 6 generates a magnetic signal of the fundamental wave f0 having the same frequency as the excitation magnetic field and a magnetic signal (high-order harmonic signal) of higher harmonics (n×f0) thereof.

A substance such as a protein that binds to a target substance contained in inspection object 6 by an antigen-antibody reaction is attached to the magnetic particle.

Zero magnetic field generator 2 forms a zero magnetic field region in the region where inspection object 6 is placed. Specifically, zero magnetic field generator 2 includes a pair of electromagnets 2a, 2b arranged to face each other such that directions of magnetization are opposite to each other. Electromagnets 2a, 2b are connected to second power supply 8a and third power supply 8b, respectively. When currents flow from second power supply 8a and third power supply 8b to electromagnets 2a, 2b, the zero magnetic field region is generated.

In the first embodiment, the case where zero magnetic field generator 2 includes electromagnets 2a, 2b will be described. However, zero magnetic field generator 2 may use two permanent magnets or a combination of a permanent magnet and an electromagnet arranged to face each other instead of electromagnets 2a, 2b. When the zero magnetic field region is formed by the two permanent magnets, second power supply 8a and third power supply 8b are omitted.

Magnetic sensor 3 detects the magnetic signal from the magnetic particle included in inspection object 6 to which the excitation magnetic field is applied. The magnetic signal indicates a change in the magnetic moment of the magnetic particle. Signal amplifier 5 amplifies the magnetic signal output from magnetic sensor 3.

Information processing device 9 is connected to each unit of detection system 100 through a bus. Information processing device 9 executes various pieces of information processing for controlling the operation of detection system 100. Information processing device 9 executes processing for selecting the magnetic particle that is applicable to in-vivo inspection and is capable of being accurately detected when bound to the target substance as a detection target magnetic particle. Furthermore, information processing device 9 acquires the magnetic signal from signal amplifier 5 and acquires a reference signal having the same frequency and phase as those of the excitation magnetic field from the first power supply. Information processing device 9 executes processing for detecting the detection target magnetic particle bound to the target substance using the magnetic signal and the reference signal.

### (Zero Magnetic Field Region)

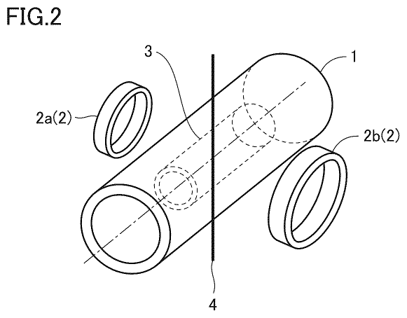

FIG. 2 is a perspective view illustrating a part of the detection system. In the example of FIG. 2, a pair of electromagnets 2a, 2b included in zero magnetic field generator 2 generates a field free line (FFL) 4. However, in the first embodiment, a shape of a zero magnetic field region 4 is not limited to the linear shape. For example, zero magnetic field region 4 may be a field free point (FFP) or may have a planar shape.

The position and direction of field free line 4 are scanned by changing a current balance of electromagnets 2a, 2b. Specifically, the distance (hereinafter, it is referred to as “translational position r”) between an origin of a coordinate system determined according to the positions of electromagnets 2a, 2b and field free line 4 and an angle (hereinafter, referred to as “angle θ”) between the axis set in the coordinate system and field free line 4 change according to the current balance of electromagnets 2a, 2b. The method for scanning zero magnetic field region 4 is not limited thereto. For example, zero magnetic field region 4 may be scanned by physical movement of electromagnets 2a, 2b. Alternatively, zero magnetic field region 4 may be relatively scanned with respect to inspection object 6 by fixing the position of zero magnetic field region 4 and moving inspection object 6.

### (Hardware Configuration of Information Processing Device)

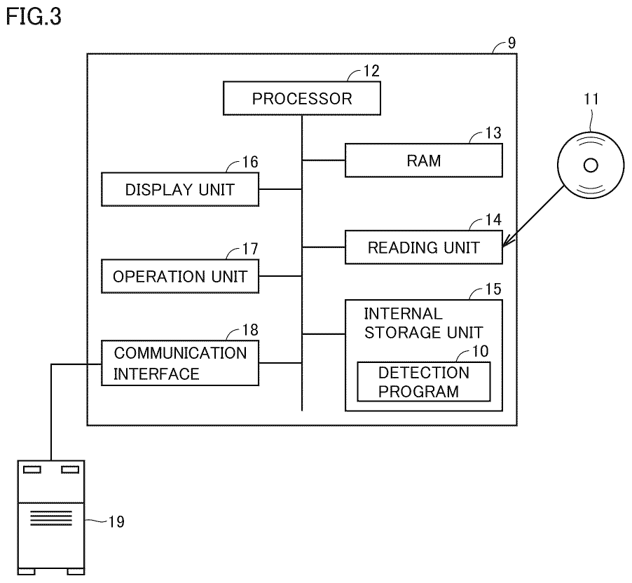

FIG. 3 is a view illustrating an example of a hardware configuration of the information processing device. As illustrated in FIG. 3, information processing device 9 includes a processor 12, a random access memory (RAM) 13, a reading unit 14, an internal storage unit 15, a display unit 16, an operation unit 17, and a communication interface 18.

For example, processor 12 is a central processing unit (CPU), and executes arithmetic processing. RAM 13 stores temporary information generated in accordance with the arithmetic processing of processor 12. Processor 12 reads a program (including detection program 10) stored in internal storage unit 15, loads the program in RAM 13, and executes the program.

Reading unit 14 reads information recorded on an optical recording medium 11 such as a compact disk read only memory (CD-ROM).

For example, internal storage unit 15 is a hard disk drive, and stores various programs such as detection program 10 and various data.

For example, display unit 16 is a liquid crystal display, and displays a screen generated according to the arithmetic processing of processor 12. For example, operation unit 17 includes a keyboard, a mouse, and the like, and receives an operation input by an operator.

Communication interface 18 communicates with an external device (for example, a server device 19) through a network.

Detection program 10 includes a command group of processing related to detection of the magnetic particle. For example, detection program 10 is recorded in optical recording medium 11, read by reading unit 14, and stored in internal storage unit 15. Alternatively, detection program 10 may be downloaded from server device 19 by communication interface 18 and stored in internal storage unit 15.

### (Flow of Magnetic Particle Detection Method)

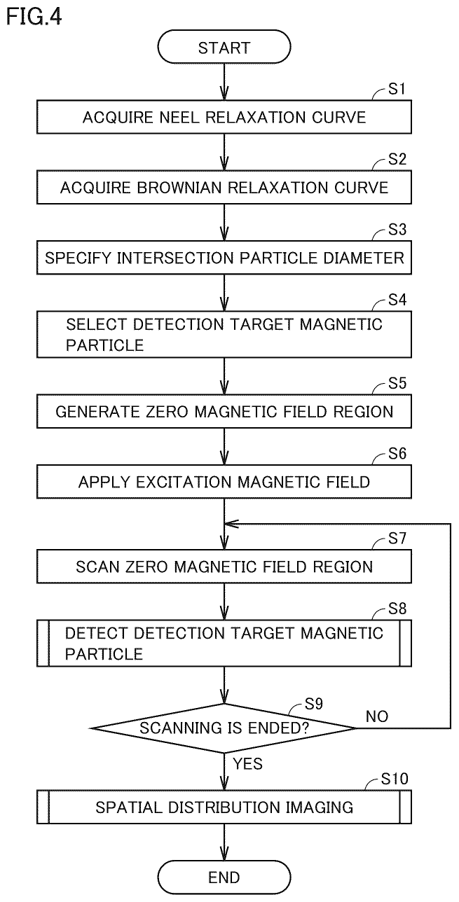

FIG. 4 is a flowchart illustrating a magnetic particle detection method of the first embodiment. The flow in FIG. 4 is executed by processor 12 according to detection program 10 loaded in RAM 13.

First, in step S1, processor 12 of information processing device 9 calculates and acquires a Neel relaxation curve indicating a relationship between a Neel relaxation time and a particle diameter for candidate magnetic particles. Further, in step S2, processor 12 calculates and obtains a Brownian relaxation curve indicating a relationship between a Brownian relaxation time and the particle diameter for the candidate magnetic particles. Subsequently, in step S3, processor 12 specifies the particle diameter corresponding to an intersection point of the Neel relaxation curve and the Brownian relaxation curve as an intersection particle diameter. In step S4, processor 12 selects a candidate magnetic particle having the particle diameter larger than the intersection particle diameter as a detection target magnetic particle.

Each of the candidate magnetic particles is a candidate for the magnetic particle provided to inspection object 6. The candidate magnetic particle is a particle capable of binding to the target substance contained in inspection object 6, and is previously designed according to the target substance.

When the volume of the magnetic particle is small, a magnetic property of the magnetic particle is easily affected by heat. Neel relaxation and Brownian relaxation are known as the influence of heat. Neel relaxation is a phenomenon in which a magnetic moment randomly rotates by heat in the magnetic particle, and as a result, the magnetization decreases. The Brownian relaxation is a phenomenon in which the magnetization decreases due to the rotation of the magnetic particle itself.

Processor 12 calculates the Neel relaxation curve indicating the relationship between a core particle radius rₙ and a Neel relaxation time τₙ using the following equations (1) and (2). τ₀ is a relaxation time constant (s), K is anisotropic energy (J/m³) of the magnetic particle, k_{B} is a Boltzmann constant (J/K), and T is a temperature (K) of the magnetic particle. Processor 12 calculates the Neel relaxation curve by inputting values input by the operator according to the candidate magnetic particles and inspection object 6 to each parameter.

$$\begin{matrix}[ Mathematical formula 1 ] & \\ {τ}_{n} = {τ}_{0} exp \frac{{KV}_{n}}{{k}_{B} T} & Equation ( 1 )\end{matrix}$$

$$\begin{matrix}[ Mathematical formula 2 ] & \\ {V}_{n} = \frac{4 π {r}_{n}^{3}}{3} & Equation ( 2 )\end{matrix}$$

Processor 12 calculates the Brownian relaxation curve indicating the relationship between a hydrodynamic radius r_{f} and a Brownian relaxation time τ_{b} using the following equations (3) and (4). Hydrodynamic radius r_{f} is a radius of a particle containing a coating outside the core of the magnetic particle, a modifying group (a protein that reacts with the target substance with an antigen-antibody), the target substance, and the like. Accordingly, when the hydrodynamic radius changes due to the configuration other than the core, an offset and inclination of the Brownian relaxation curve change. η is viscosity (Js/m³) of the medium in which the magnetic particle exists. Processor 12 calculates the Brownian relaxation curve by inputting values input by the operator according to the candidate magnetic particles and inspection object 6 to each parameter.

$$\begin{matrix}[ Mathematical formula 3 ] & \\ {τ}_{b} = \frac{3 η {V}_{f}}{{k}_{B} T} & Equation ( 3 )\end{matrix}$$

$$\begin{matrix}[ Mathematical formula 4 ] & \\ {V}_{f} = \frac{4 π {r}_{f}^{3}}{3} & Equation ( 4 )\end{matrix}$$

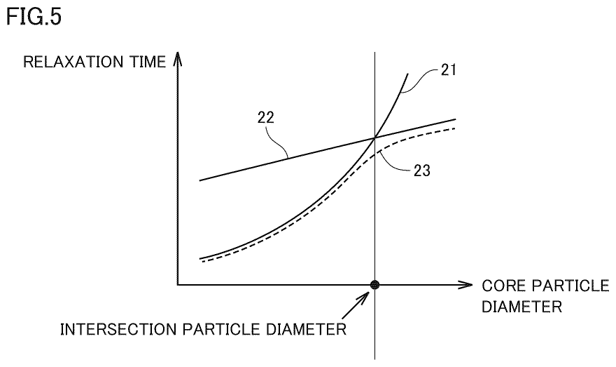

FIG. 5 is a view illustrating examples of a Neel relaxation curve and a Brownian relaxation curve. The horizontal axis of FIG. 5 indicates a core particle diameter as a particle diameter converted from core particle radius rₙ and hydrodynamic radius r_{f}. Processor 12 may convert core particle radius rₙ of the Neel relaxation curve calculated from equations (1) and (2) into the core particle diameter. Similarly, processor 12 may convert hydrodynamic radius r_{f} of the Brownian relaxation curve calculated from equations (3) and (4) into the core particle diameter.

As illustrated in FIG. 5, slope of a Neel relaxation curve 21 is greater than slope of a Brownian relaxation curve 22, and the Neel relaxation time is less than the Brownian relaxation time when the core particle diameter is small. Consequently, Neel relaxation curve 21 and Brownian relaxation curve 22 intersect. Processor 12 specifies the core particle diameter corresponding to the intersection of Neel relaxation curve 21 and Brownian relaxation curve 22 as an intersection particle diameter.

An effective relaxation time of the magnetization of the magnetic particle follows the shorter one of the Neel relaxation time and the Brownian relaxation time. In FIG. 5, an effective relaxation curve 23 indicates the relationship between the core particle diameter and the effective relaxation time in the candidate magnetic particles. As illustrated by effective relaxation curve 23, the magnetization is relaxed according to the Neel relaxation time for the candidate magnetic particle having the core particle diameter smaller than the intersection particle diameter, and the magnetization is relaxed according to the Brownian relaxation time for the candidate magnetic particle having the core particle diameter larger than the intersection particle diameter.

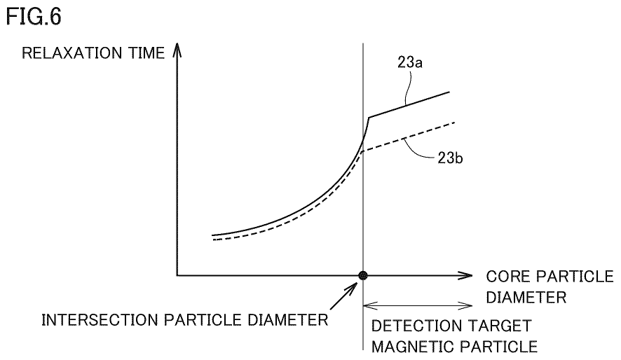

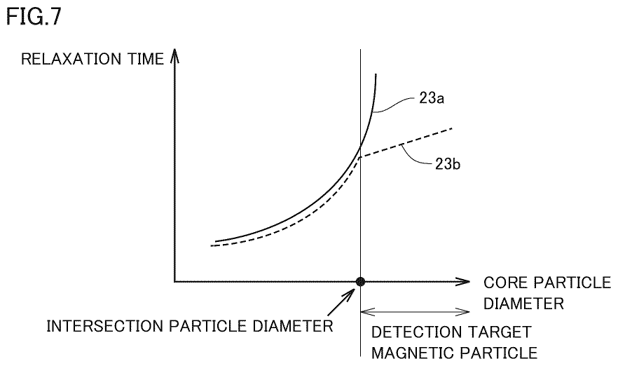

FIG. 6 is a view illustrating an example of the effective relaxation curves of a bound particle and an unbound particle. FIG. 7 is a view illustrating another example of the effective relaxation curves of the bound particle and the unbound particle. In FIGS. 6 and 7, a reference numeral 23a indicates the effective relaxation curve of the candidate magnetic particles (bound particles) to which the target substances are bound. A reference numeral 23b indicates the effective relaxation curve of the candidate magnetic particles (unbound particles) to which the target substances are not bound. FIG. 6 illustrates effective relaxation curves 23a, 23b when the candidate magnetic particles are rotatable even after bound to the target substances. FIG. 7 illustrates effective relaxation curves 23a, 23b when the candidate magnetic particles are not rotatable even after bound to the target substances.

As illustrated in FIGS. 6 and 7, at the core particle diameter smaller than the intersection particle diameter, there is almost no difference between effective relaxation curve 23a of the bound particles and effective relaxation curve 23b of the unbound particles. On the other hand, when the core particle diameter is larger than the intersection particle diameter, the difference between effective relaxation curve 23a of the bound particles and effective relaxation curve 23b of the unbound particles becomes large. That is, the relaxation time of the candidate magnetic particle having the core particle diameter larger than the intersection particle diameter varies depending on whether the candidate magnetic particle is bound to the target substance. Accordingly, when the excitation magnetic field is applied to the candidate magnetic particle having the core particle diameter larger than the intersection particle diameter, the phase of the magnetic signal from the candidate magnetic particle varies depending on whether the magnetic signal is bound to the target substance. That is, the use of phase information can discriminate between the bound particle and the unbound particle. Accordingly, as illustrated in FIGS. 6 and 7, processor 12 selects the candidate magnetic particle having the core particle diameter larger than the intersection particle diameter as the detection target magnetic particle.

Returning to FIG. 4, the processing after step S5 will be described. In step S5, processor 12 generates a command for controlling the power supply to electromagnets 2a, 2b, and outputs the generated command to second power supply 8a and third power supply 8b. Thus, second power supply 8a and third power supply 8b start the power supply to electromagnets 2a, 2b in response to the command. As a result, a zero magnetic field region is generated in inspection object 6. The candidate magnetic particle is injected into inspection object 6.

Subsequently, in step S6, processor 12 generates the command for controlling the power supply to excitation magnetic field applicator 1, and outputs the generated command to first power supply 7. Thus, first power supply 7 starts the power supply to excitation magnetic field applicator 1 in response to the command. As a result, the AC excitation magnetic field is applied to inspection object 6.

Subsequently, in step S7, processor 12 scans the zero magnetic field region in inspection object 6 by adjusting the current balance from second power supply 8a and third power supply 8b to electromagnets 2a, 2b. When the zero magnetic field region is located at the first scanning position in step S5, first step S7 is omitted.

Subsequently, in step S8, processor 12 detects a change in the magnetic moment of the detection target magnetic particle due to the excitation magnetic field, and stores the detection result.

Subsequently, in step S9, processor 12 determines whether the scanning of the zero magnetic field region in inspection object 6 is ended. When the scanning is not ended (NO in step S9), the processing returns to step S7. Thus, step S7 and step S8 are performed for each scanning position in the zero magnetic field region.

When the scanning is completed (YES in step S9), in step S10, processor 12 executes processing (spatial distribution imaging) for generating the image indicating a spatial distribution in which the target substance exists in inspection object 6 using the stored detection result.

The order of step S5 and step S6 may be reversed. In addition, the order of step S7 and step S8 may be reversed.

### (Subroutine of Step S8)

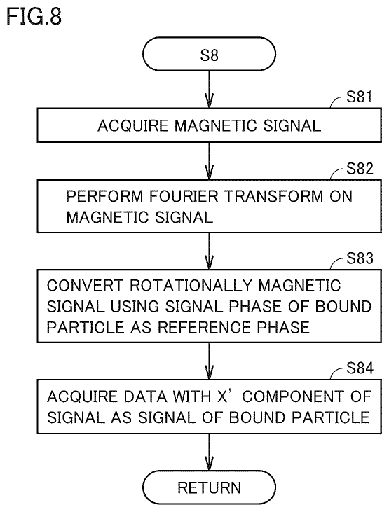

FIG. 8 is a flowchart illustrating a flow of a subroutine of step S8 in FIG. 4. As illustrated in FIG. 8, in step S81, processor 12 acquires, from signal amplifier 5, the magnetic signal indicating the change in the magnetic moment of the detection target magnetic particle existing in the zero magnetic field region according to the excitation magnetic field. Subsequently, in step S82, processor 12 performs Fourier transform on the magnetic signal. Most of the fundamental wave signal is due to the excitation magnetic field. Accordingly, in step S82, processor 12 preferably detects the phase of the high-order harmonic signal generated according to the change in the magnetic moment.

Subsequently, in step S83, processor 12 rotationally converts the magnetic signal using the signal phase of the bound particle as a reference phase. In step S84, processor 12 acquires the component of the reference phase in the rotationally transformed magnetic signal as the signal of the bound particle. That is, processor 12 determines the presence or absence of binding between the detection target magnetic particle and the target substance based on the phase of the magnetic signal, and acquires the signal of the bound particle. Processor 12 stores the acquired signal of the bound particle and the information (translational position r and angle θ) indicating the scanning position of the zero magnetic field region in association with each other.

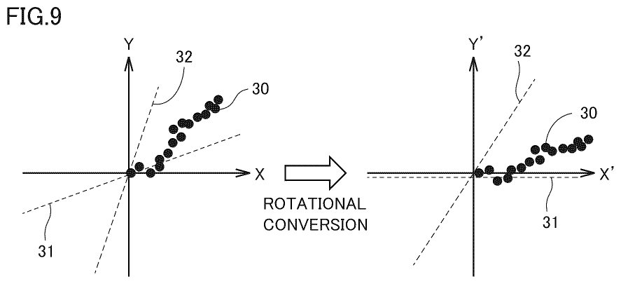

FIG. 9 is a view illustrating processing contents of steps S83, S84. In FIG. 9, an X-axis represents a component following the AC excitation magnetic field in the change in the magnetic moment of the detection target magnetic particle. A Y-axis indicates a delay component with respect to the AC excitation magnetic field in the change in the magnetic moment of the detection target magnetic particle. The delay component is shifted by 90° with respect to the following component.

A left side of FIG. 9 illustrates a state in which a magnetic signal 30 after the Fourier transform is plotted on an XY-plane. A signal phase 31 of the bound particle and a signal phase 32 of the unbound particle are previously measured and registered in information processing device 9. Information processing device 9 rotationally converts magnetic signal 30 such that signal phase 31 of the bound particle becomes the reference phase. Thus, the X-axis is rotationally transformed to an X′-axis, and the Y-axis is rotationally transformed to a Y′-axis. Processor 12 may calculate signal phase 31 of the bound particles from the relaxation time of effective relaxation curve 23a of the bound particles in FIGS. 6 and 7, and may calculate a rotation transformation matrix according to the calculation result.

Processor 12 obtains an X′-axis component of rotationally transformed magnetic signal 30 as the signal of the bound particle.

### (Subroutine of Step S10)

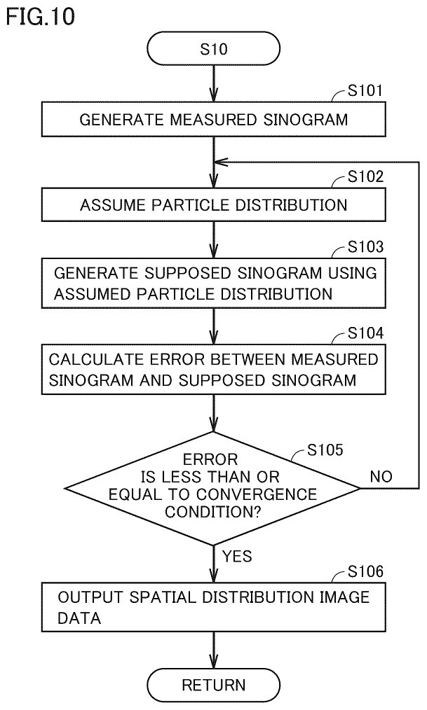

FIG. 10 is a flowchart illustrating a flow of a subroutine of step S10 in FIG. 4. FIG. 10 illustrates a method for generating the image illustrating the spatial distribution of the bound particle using a known successive approximation image reconstruction method.

As illustrated in FIG. 10, in step S101, processor 12 generates a sinogram (hereinafter, referred to as “measured sinogram”) from the signal of the bound particle and the information indicating the scanning position of the zero magnetic field region that are stored in step S8. The sinogram is a signal map in which a horizontal axis is angle θ and a vertical axis is translational position r.

Subsequently, in step S102, processor 12 assumes a distribution of the bound particle. In step S103, processor 12 generates a supposed sinogram using the distribution assumed in step S102. In step S104, processor 12 calculates an error between the measured sinogram generated in step S101 and the supposed sinogram generated in step S103. In step S105, processor 12 determines whether the error is less than or equal to a predetermined convergence condition. When the negative determination is made in step S105, the processing returns to step S102.

Processor 12 repeats the pieces of processing from step S102 to step S104 until the error becomes less than or equal to the convergence condition.

When the affirmative determination is made in step S105, in step S106, processor 12 generates data (spatial distribution image data) indicating the image indicating the spatial distribution of the bound particle corresponding to the supposed sinogram satisfying the convergence condition, and outputs the generated data. For example, processor 12 causes display unit 16 to display the image indicating the spatial distribution of the bound particle.

As described in “R. Matthew Ferguson et al., “Optimization of nanoparticle core size for magnetic particle imaging” J. Magn. Magn. Mater., 321 (2009), pp 1548-1551” (NPL 1), in the conventional magnetic particle imaging device, the excitation frequency and the core size of the magnetic particle are generally selected so as to minimize the influence of the relaxation delay. However, even when the signal intensity slightly decreases due to the relaxation delay, the spatial distribution of the bound particle can be imaged by discriminating the bound particle and the unbound particle from the phase of the magnetic signal. Thus, contrast of the image can be improved.

At this point, the case where zero magnetic field region 4 is linear has been described. However, as described above, the shape of the zero magnetic field region is not limited to the linear shape. In the case where the shape of zero magnetic field region 4 is not linear, using information indicating the correspondence between the scanning position of zero magnetic field region 4 and the signal intensity at the scanning position, processing for determining the assumed distribution may be performed such that the error between the assumed value obtained from the assumed distribution and the measured value is less than or equal to the convergence condition.

### Second Embodiment

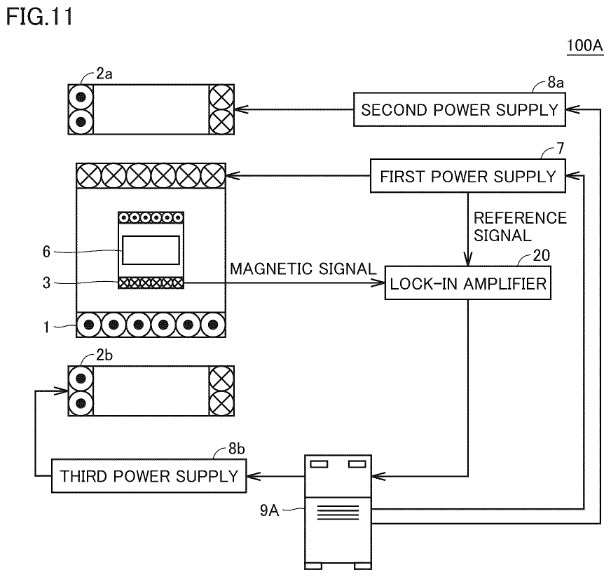

FIG. 11 is a view illustrating an example of an overall configuration of a detection system according to a second embodiment. As illustrated in FIG. 11, a detection system 100A of the second embodiment is different from detection system 100 of the first embodiment in that detection system 100A includes a lock-in amplifier 20 and an information processing device 9A instead of signal amplifier 5 and information processing device 9.

Lock-in amplifier 20 extracts a signal having a known frequency and phase from the input signal. The magnetic signal measured by magnetic sensor 3 is input to lock-in amplifier 20 as the input signal. Furthermore, the reference signal having the same frequency and phase as the AC excitation magnetic field is input from first power supply 7 to lock-in amplifier 20. Lock-in amplifier 20 adjusts the phase of the reference signal to be matched with the phase of the magnetic signal from the bound particle according to a predetermined setting. Lock-in amplifier 20 extracts a high-order harmonic signal having a phase specific to the bound particle from the magnetic signal measured by magnetic sensor 3 by performing synchronous detection of the input signal and the adjusted reference signal, and outputs the extracted signal to information processing device 9A.

Information processing device 9A has a hardware configuration similar to that of information processing device 9 of the first embodiment. Similarly to the first embodiment, processor 12 executes processing according to the flowchart in FIG. 4.

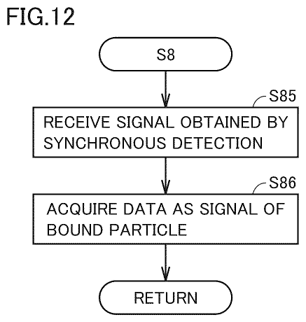

FIG. 12 is a flowchart illustrating a flow of the subroutine of step S8 in FIG. 4 of the second embodiment.

As illustrated in FIG. 12, in step S85, processor 12 receives the signal obtained by synchronous detection of lock-in amplifier 20. As described above, the signal is a high-order harmonic signal having the phase specific to the bound particle. Subsequently, in step S86, processor 12 acquires the signal received in step S85 as the signal of the bound particle.

### Third Embodiment

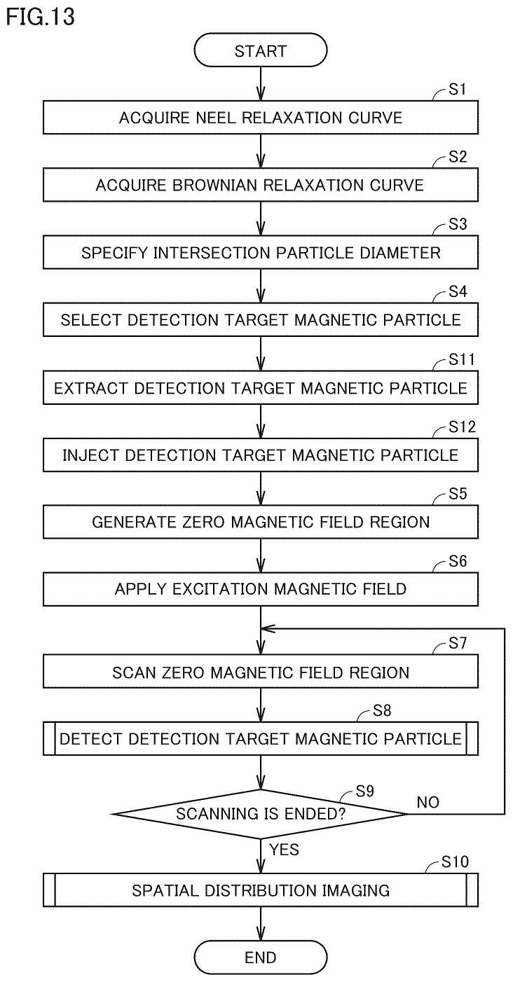

FIG. 13 is a flowchart illustrating a flow of processing of a detection method in a third embodiment. The flowchart in FIG. 13 is different from the flowchart in FIG. 4 in that step S11 and step S12 are included.

As illustrated in FIG. 13, in step S11 after step S4, in order to reduce the magnetic particle having the core particle diameter smaller than the intersection particle diameter, the detection target magnetic particle having the core particle diameter larger than the intersection particle diameter is extracted from among candidate magnetic particles.

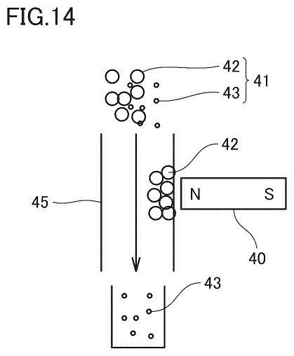

FIG. 14 is a view illustrating an example of a device executing step S11. As illustrated in FIG. 14, the device includes a column 45 passing candidate magnetic particles 41 and a permanent magnet 40 disposed outside column 45.

Candidate magnetic particles 41 include a detection target magnetic particle 42 having a core particle diameter larger than the intersection particle diameter, and a non-target magnetic particle 43 having the core particle diameter smaller than the intersection particle diameter. Because detection target magnetic particle 42 is more easily magnetized, detection target magnetic particle 42 receives larger force of the magnetic field. Accordingly, when candidate magnetic particles 41 are input to column 45, detection target magnetic particle 42 is attracted to the magnetic field, and non-target magnetic particle 43 passes through column 45. Thus, detection target magnetic particle 42 and the non-target magnetic particle are separated, and detection target magnetic particle 42 is extracted. Instead of the permanent magnet, an electromagnet including a coil and a magnetic body may be used. Alternatively, detection target magnetic particle 42 may be physically extracted using a mesh sieve.

As illustrated in FIG. 13, in step S12 after step S11, extracted detection target magnetic particle 42 is injected into inspection object 6. After step S12, the same steps S5 to S10 as in FIG. 4 are performed.

The signal of the candidate magnetic particle having the core particle diameter smaller than the intersection particle diameter has the same phase regardless of whether the candidate magnetic particle is bound to the target substance. Consequently, the candidate magnetic particle having the core particle diameter smaller than the intersection particle diameter cannot be used for discrimination between bound particle and unbound particle. When the candidate magnetic particle having the core particle diameter smaller than the intersection particle diameter is reduced from the candidate magnetic particles, a ratio at which an extra signal that does not contribute to discrimination is input to signal amplifier 5 or lock-in amplifier 20 can be reduced. P Thus, the signal by the detection target magnetic particle can be further amplified, and S/N is improved.

### Modifications

In the above description, it is assumed that the detection system generates the image illustrating the spatial distribution of the bound particle. However, in the case of the total amount inspection that does not require the spatial distribution imaging, steps S5 and S7 to S10 in FIG. 4 can be omitted.

It should be considered that the disclosed embodiments are an example in all respects and not restrictive. The scope of the present disclosure is defined by not the description of the embodiments, but the claims, and it is intended that all changes within the meaning and scope of the claims are included in the present invention.

### REFERENCE SIGNS LIST

1: excitation magnetic field applicator, 2: zero magnetic field generator, 2a, 2b: electromagnet, 3: magnetic sensor, 4: zero magnetic field region, 5: signal amplifier, 6: inspection object, 7: first power supply, 8a: second power supply, 8b: third power supply, 9, 9A: information processing device, 10: detection program, 11: optical recording medium, 12: processor, 13: RAM, 14: reading unit, 15: internal storage unit, 16: display unit, 17: operation unit, 18: communication interface, 19: server device, 20: lock-in amplifier, 21: Neel relaxation curve, 22: Brownian relaxation curve, 23, 23a, 23b: effective relaxation curve, 40: permanent magnet, 41: candidate magnetic particles, 42: detection target magnetic particle, 43: non-target magnetic particle, 45: column, 100, 100A: detection system

1. A detection method for detecting a detection target magnetic particle using an AC excitation magnetic field, the detection method comprising:
acquiring a first curve indicating a relationship between a Neel relaxation time and a particle diameter for candidate magnetic particles;
acquiring a second curve indicating a relationship between a Brownian relaxation time and the particle diameter for the candidate magnetic particles;
specifying a particle diameter corresponding to an intersection of the first curve and the second curve as an intersection particle diameter; and
selecting a candidate magnetic particle having a particle diameter larger than the intersection particle diameter as the detection target magnetic particle.

2. The detection method according to claim 1, further comprising:
applying the excitation magnetic field to the detection target magnetic particle; and detecting a change in magnetic moment of the detection target magnetic particle due to the excitation magnetic field.

3. The detection method according to claim 2, wherein
the detection target magnetic particle is capable of binding to a target substance, and
the detecting includes:
detecting a phase of a high-order harmonic signal generated in response to the change in the magnetic moment; and
determining presence or absence of binding between the detection target magnetic particle and the target substance based on the phase.

4. The detection method according to claim 3, further comprising:
generating a zero magnetic field region in an inspection object in which the detection target magnetic particle and the target substance exist;
scanning the zero magnetic field region in the inspection object; and
generating an image indicating a spatial distribution of the detection target magnetic particle determined to be bound to the target substance in the inspection object.

5. The detection method according to claim 1, further comprising:
extracting the detection target magnetic particle from the candidate magnetic particles; and
injecting the detection target magnetic particle extracted in the extracting into an inspection object in which a target substance capable of binding to the detection target magnetic particle exists.

6. A detection system that detects a detection target magnetic particle using an excitation magnetic field, the detection system comprising a processor to execute information processing for selecting the detection target magnetic particle from candidate magnetic particles,
wherein the processor
acquires a first curve indicating a relationship between a Neel relaxation time and a particle diameter for the candidate magnetic particles;
acquires a second curve indicating a relationship between a Brownian relaxation time and a particle diameter for the candidate magnetic particles;
specifies a particle diameter corresponding to an intersection of the first curve and the second curve as an intersection particle diameter, and
selects a candidate magnetic particle having a particle diameter larger than the intersection particle diameter as the detection target magnetic particle.

7. The detection system according to claim 6, further comprising:
an applicator to apply the excitation magnetic field to the detection target magnetic particle; and
a sensor to detect a magnetic signal indicating a change in magnetic moment of the detection target magnetic particle due to the excitation magnetic field.

8. The detection system according to claim 7, wherein
the detection target magnetic particle is capable of binding to a target substance, and
the processor further detects a phase of a high-order harmonic signal generated in response to the change in the magnetic moment based on the magnetic signal; and determines presence or absence of binding between the detection target magnetic particle and the target substance based on the phase.

9. The detection system according to claim 7, wherein
the detection target magnetic particle is capable of binding to a target substance,
the detection system further includes a lock-in amplifier to extract a high-order harmonic signal having a phase corresponding to the detection target magnetic particle bound to the target substance from the magnetic signal, and
the processor determines presence or absence of binding between the detection target magnetic particle and the target substance based on the high-order harmonic signal.

10. The detection system according to claim 8, further comprising:
a zero magnetic field generator to generate a zero magnetic field region in an inspection object in which the detection target magnetic particle and the target substance exist; and
a scanning unit to scan the zero magnetic field region in the inspection object,
wherein the processor generates an image indicating a spatial distribution of the detection target magnetic particle determined to be bound to the target substance in the inspection object based on a scanning position of the zero magnetic field region and a determination result of the presence or absence of the binding.

11.-13. (canceled)

14. A non-transitory computer-readable recording medium on which a computer program is recorded, the computer program supporting a detection system detecting a detection target magnetic particle using an excitation magnetic field, the computer program causing a computer to execute:
acquiring a first curve indicating a relationship between a Neel relaxation time and a particle diameter for candidate magnetic particles;
acquiring a second curve indicating a relationship between a Brownian relaxation time and the particle diameter for the candidate magnetic particles;
specifying a particle diameter corresponding to an intersection of the first curve and the second curve as an intersection particle diameter; and
selecting a candidate magnetic particle having the particle diameter larger than the intersection particle diameter as the detection target magnetic particle.

15. The non-transitory computer-readable recording medium according to claim 14, wherein
the detection target magnetic particle is capable of binding to the target substance,
the computer program further causing the computer to execute determining presence or absence of binding between the detection target magnetic particle and the target substance based on a phase of a high-order harmonic signal generated in response to a change in magnetic moment of the detection target magnetic particle due to the excitation magnetic field.

16. The non-transitory computer-readable recording medium according to claim 15, wherein
the detection system includes:
a zero magnetic field generator to generate a zero magnetic field region in an inspection object in which the detection target magnetic particle and the target substance exist; and
a scanning unit to scan the zero magnetic field region in the inspection object,
the computer program further causing the computer to execute generating an image indicating a spatial distribution of the detection target magnetic particle determined to be bound to the target substance in the inspection object based on a scanning position of the zero magnetic field region and a determination result of the presence or absence of the binding.

17. The detection method according to claim 2, further comprising:
extracting the detection target magnetic particle from the candidate magnetic particles; and
injecting the detection target magnetic particle extracted in the extracting into an inspection object in which a target substance capable of binding to the detection target magnetic particle exists.

18. The detection system according to claim 9, further comprising:
a zero magnetic field generator to generate a zero magnetic field region in an inspection object in which the detection target magnetic particle and the target substance exist; and
a scanning unit to scan the zero magnetic field region in the inspection object,
wherein the processor generates an image indicating a spatial distribution of the detection target magnetic particle determined to be bound to the target substance in the inspection object based on a scanning position of the zero magnetic field region and a determination result of the presence or absence of the binding.
